(19) United States
(12) Patent Application Publication
(10) Pub. No.: US 2012/0232805 A1
(43) Pub. Date: Sep. 13, 2012

(54) Computerized Amino Acid Composition Enumeration

(75) Inventors: Rovshan G. Sadygov, League City, TX (US); Indranil Mitra, Houston, TX (US)

(73) Assignee: Board of Regents, The University of Texas System, Austin, TX (US)

(21) Appl. No.: 13/396,340

(22) Filed: Feb. 14, 2012

Related U.S. Application Data
(60) Provisional application No. 61/463,295, filed on Feb. 14, 2011.

(51) Int. Cl. G06F 19/20; H01J 49/40; H01J 49/26; G06F 19/24; G01N 33/483

(52) U.S. Cl. 702/20; 702/19

(57) ABSTRACT

A computerized method and apparatus for enumerating one or more amino acid compositions is disclosed that provides one or more processors, a data storage communicably coupled to the one or more processors and a user interface communicably coupled to the one or more processors. The three or more user-specified characteristics are received from the data storage or the user interface. The one or more amino acid compositions are enumerated for all the peptides having a length less than or equal to the maximum length and a mass less than or equal to the mass limit using the one or more processors. The enumerated amino acid compositions are filtered based on the one or more other user-specified characteristics using the one or more processors. The filtered amino acid compositions and the mass of the filtered amino acid compositions are stored in the data storage.

### CROSS-REFERENCE TO RELATED APPLICATIONS

This application claims priority to U.S. Provisional Application Ser. No. 61/463,295 filed, Feb. 14, 2011, the entire contents of which are incorporated herein by reference.

### STATEMENT OF FEDERALLY FUNDED RESEARCH

This invention was made with U.S. Government support under Contract No. HHSN272200800048C from NIAID and Contract No. NIH-NLBIHHSN268201000037C from NHLBI of the NIH. The government has certain rights in this invention.

### TECHNICAL FIELD OF THE INVENTION

The present invention relates in general to the field of amino acid analysis, and more particularly, to computerized amino acid composition enumeration.

### INCORPORATION-BY-REFERENCE OF MATERIALS FILED ON COMPACT DISC

None.

### BACKGROUND OF THE INVENTION

Without limiting the scope of the invention, its background is described in connection with the determination of amino acid compositions.

Peptides are made of twenty amino acids with specific masses, which leads to an inhomogeneous, clustered distribution of their masses. This can be observed both for all theoretically possible peptides, and for real protein sequence databases. In the distribution, peptides form a series of peaks separated by approximately 1 Da, which grow taller and wider as the mass increases. Consecutive peaks are separated by low populated areas (quiet zones) and gaps (forbidden zones)—that is, the mass ranges for which there exist no possible sequences of amino acids.

These features of the distribution of peptide masses play important role in the mass spectrometry-based proteomics. Forbidden zones are used to filter out non-peptide masses in a variety of experiments and workflows, including peptide mass fingerprinting [1] and mass defect filtering [2]. They also find application in recently proposed mass defect labeling [3-5]. A very promising research is being done in de novo peptide sequencing based on highly accurate mass measurements and the discrete nature of the distribution of peptide masses [6-9].

A recent work by Mickalski [10] and colleagues has suggested a practical way of separating peptide species from co-eluting contaminants. It was observed that in an equal mixture of completely labeled and label-free SILAC [11] samples all peptide species would have isotopically labeled counterparts. Absence of a labeled counterpart indicates that the species is non-peptidic. It was observed that up to 30% of all eluting species were of non-peptide origin. Ability to filter out these species, or even better not to choose them for fragmentation in the first place, will significantly improve the efficiency of experiments.

Thus, an increasing number of proteomics workflows face the need for efficient generation and characterization of mass distributions of different classes of peptides. Often, it is also desirable to permanently store these distributions (along with the atomic, amino acid, or other compositions of the molecules) and use them as look up tables. Since the number of all sequences comprised of N letters and having length L is equal to N^{L}, the main challenge here is a high computational cost associated with generation of these distributions and huge amounts of data to be stored.

One of the pioneering works on generation and description of the mass distribution of all theoretically possible peptides was done by Mann in 1995 [12]. He considered the mass range up to 2 kDa and suggested linear equations linking nominal peptide masses with the position and width of the peaks formed by monoisotopic peptide masses. He described a range of potential applications of his results, including the possibility of the use of suggested equations to identify non-peptide masses. It was noted that the computational time needed to generate a 50 Da wide range of the distribution was less than one second for masses below 500 Da and 18 hours for masses around 2 kDa.

Since then, a number of papers were published in which theoretical or observed distributions of peptide masses were generated [1,8,13-17] for different proteomics applications. These distributions usually covered the mass range up to 2 kDa, which did not allow full characterization of the forbidden zones. Also, their generation often required long computational times or/and extensive computational capabilities. In many cases, the distributions were not made easily available for the research community for independent validation, study and use.

U.S. Pat. No. 6,489,608, issued to Skilling, teaches a method of determining peptide sequences by mass spectrometry. Briefly, a method of determining the sequence of amino acids, e.g., peptides, polypeptides or proteins by mass spectrometry and especially by tandem mass spectrometry is disclosed. The method is said to work without the use of any additional data concerning the nature of the peptide and without any limit to the number of possible sequences considered. The method can be implemented on a personal computer typically used for data acquisition on the tandem mass spectrometer even in the case of peptides comprising 10 or more amino acids. The method does not rely on exhaustive comparison of the spectra predicted from every possible amino acid sequence with any molecular weight constraint, but instead uses mathematical techniques to simulate the effect of such a complete search without actually carrying it out.

Another such method is taught by Ma, et al., using a system entitled “PEAKS: powerful software for peptide de novo sequencing by tandem mass spectrometry,” Rapid Commun. Mass Spectrom. 2003; 17: 2337-2342, which describes a de novo sequencing software package, PEAKS, used to extract amino acid sequence information without the use of databases. PEAKS is said to efficiently compute the best peptide sequences whose fragment ions can best interpret the peaks in the MS/MS spectrum. The output of the software gives amino acid sequences with confidence scores for the entire sequences, as well as an additional novel positional scoring scheme for portions of the sequences. The performance of PEAKS was compared with Lutefisk, a well-known de novo sequencing software, using quadrupole-time-of-flight (Q-TOF) data obtained for several tryptic peptides from standard proteins.

### SUMMARY OF THE INVENTION

The present invention provides a computerized method of enumerating one or more amino acid compositions by providing one or more processors, a data storage communicably coupled to the one or more processors and a user interface communicably coupled to the one or more processors. The three or more user-specified characteristics are received from the data storage or the user interface. The three or more user-specified characteristics include a mass limit for the amino acid compositions, a maximum length for the amino acid compositions, and one or more other user-specified characteristics. The one or more amino acid compositions are enumerated for all the peptides having a length less than or equal to the maximum length and a mass less than or equal to the mass limit using the one or more processors. The enumerated amino acid compositions are filtered based on the one or more other user-specified characteristics using the one or more processors. The filtered amino acid compositions and the mass of the filtered amino acid compositions are stored in the data storage. The foregoing method can be implemented as a non-transitory computer-readable medium wherein the steps are executed as one or more code segments by one or more processors.

The present invention also provides an apparatus for enumerating one or more amino acid compositions. The apparatus includes one or more processors, a data storage communicably coupled to the one or more processors, and a user interface communicably coupled to the one or more processors. The one or more processors (a) receive the three or more user-specified characteristics from the data storage or the user interface, (b) enumerate the one or more amino acid compositions for all the peptides having a length less than or equal to the maximum length and a mass less than or equal to the mass limit using the one or more processors, (c) filter the enumerated amino acid compositions based on the one or more other user-specified characteristics, and (d) store the filtered amino acid compositions and the mass of the filtered amino acid compositions in the data storage. The three or more user-specified characteristics may include a mass limit for the amino acid compositions, a maximum length for the amino acid compositions, and one or more other user-specified characteristics.

### BRIEF DESCRIPTION OF THE DRAWINGS

For a more complete understanding of the features and advantages of the present invention, reference is now made to the detailed description of the invention along with the accompanying figures and in which:

FIG. 1 is a graph showing the logarithmic plot of the number of tryptic peptide compositions with masses up to 3 kDa (separate plots for all lengths between 2 and 51, and five lengths from 5 to 25).

FIG. 2 is a graph showing the number of peptide compositions for three mass ranges.

FIG. 3 is a graph showing the coefficient of skewness calculated for peaks in the mass distribution of all theoretically possible tryptic peptides.

FIG. 4 is a graph showing the width of gaps for different mass ranges.

FIG. 5 is a graph showing the width of gaps between peaks of the mass distribution.

FIG. 6 are graphs showing a fragment of the non-restricted mass distribution with red peaks corresponding to prohibited compositions, making up 18% of all compositions (top graph) and wherein the red peaks correspond to uniformly chosen compositions making up 18% of all compositions (bottom graph).

FIG. 7 are graphs showing a fragment of the non-restricted mass distribution with red peaks corresponding to prohibited compositions (according to restrictions derived from the IPI human proteome database) making up 45% of all compositions (top graph), and red peaks correspond to uniformly chosen 45% of all compositions (bottom graph).

FIG. 8 is a graph showing the width of gaps between peaks of the mass distribution.

FIG. 9 is graph showing the average entropy (red) and the margin of three standard deviations (aquamarine) plotted against the number of compositions (black) for the mass range 1900.25-1901.50 Da.

FIG. 10 shows creating multiple jobs from a single job of enumerating all compositions.

FIG. 11 shows a flow chart of a computerized method of enumerating one or more amino acid compositions in accordance with one embodiment of the present invention.

FIG. 12 shows a block diagram of an apparatus for enumerating one or more amino acid compositions in accordance with another embodiment of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined herein. Terms defined herein have meanings as commonly understood by a person of ordinary skill in the areas relevant to the present invention. The terminology herein is used to describe specific embodiments of the invention, but their usage does not delimit the invention, except as outlined in the claims.

The present invention will be described using an example with the mass distribution of all theoretically possibly tryptic peptides made of 20 amino acids, up to the mass of 3 kDa, with accuracy of 0.001 Da. Note that the present invention is not limited to tryptic peptides, or peptides made of 20 amino acids, or peptides having a mass up to 3 kDa, or an accuracy of 0.001 Da. The regions are characterized between the peaks of the distribution, including gaps (forbidden zones) and low-populated areas (quiet zones). The gaps shrink over the mass range, and then they completely disappear. Peptide compositions in quiet zones are less diverse than those in the peaks of the distribution, and that by eliminating certain types of unrealistic compositions the gaps in the distribution may be increased. The mass distribution is generated using a parallel implementation of a recursive procedure that enumerates all amino acid compositions. As a result, all compositions of tryptic peptides below 3 kDa can be enumerated in 48 minutes using a computer cluster with 12 Intel Xeon X5650 CPUs (72 cores). The present invention can be used to facilitate protein identification and mass defect labeling in mass spectrometry-based proteomics experiments.

The mass distribution is generated using a parallel implementation of a recursive procedure that enumerates all amino acid (AA) compositions. Suggested parallel implementation of the procedure yields significant reduction in the computation time. For instance, the distribution described herein was generated in 48 minutes on a computer cluster with 12 Intel Xeon X5650 CPUs (72 cores). Given the fact that parallel computing architectures become increasingly available, this result opens new perspectives on the use of brute-force enumeration of amino acids compositions in proteomics studies. In particular, if peptide mass distributions can be generated quickly, then they can be generated “to order” to facilitate protein identification in particular experiments or experimental setups, taking into account specific proteases used in sample preparation, possible post-translational or chemical modifications, and other factors.

A peptide may be considered as a sequence of letters from a 20-letter alphabet A={a₁, a₂, . . . , a₂₀}, whose letters correspond to 20 amino acids. This sequence can be coded by a numerical vector (n₁, n₂, . . . , n₂₀), whose i-th component is the number of occurrences of the i-th letter (amino acid) in the sequence, i=1, 2, . . . , 20. This vector is called a composition (also amino acid composition or peptide composition). For example, sequence a₁a₂₀a₁a₁ results in composition (3, 0, . . . , 0, 1). The present invention generates all theoretically possibly peptide masses by using amino acid compositions and a recursive procedure for enumerating these compositions, which is executed in parallel.

It is worthwhile to note some basic properties of compositions. For the sake of generality, consider alphabet A of N letters a₁, a₂, . . . , a_{N}, and composition (n₁, n₂, . . . , n_{N}). The length of the composition is defined as L=n₁+n₂+ . . . +n_{N}. For a single sequence of letters a uniquely defined composition is provided, while for a single composition of length L there are

$$\frac{L !}{{n}_{1} ! {n}_{2} ! … {n}_{N} !} \qquad ( 1 )$$

corresponding sequences, since the composition codes only the number of letters used in the sequence but not their order. The number of compositions of length L that use N letters is equal to the number of ways to choose L elements from a set of N elements if repetitions are allowed:

$$( \begin{matrix}N + L - 1 \\ L\end{matrix} ) = \frac{( N + L - 1 ) !}{L ! ( N - 1 ) !} , \qquad ( 2 )$$

while the number of corresponding sequences is equal to N^{L}. Table 1 shows the number of compositions and sequences for all peptides and tryptic peptides up to the length 10. Note how the number of sequences quickly overgrows the number of compositions as L increases.

TABLE 1
Number of compositions and sequences corresponding to all peptides
and tryptic peptides of length 3-10.

| | | | Tryptic peptides | |
|---|---|---|---|---|
| | All peptides | | (18 AA, K or R in the end) | |
| | (20 AA) | | Number of | |
| Peptides' | Number of | Number of | com- | Number of |
| length (L) | compositions | sequences | positions | sequences |
| 3 | 1,540 | 8,000 | 342 | 648 |
| 4 | 8,855 | 160,000 | 2,280 | 11,664 |
| 5 | 42,504 | 3,200,000 | 11,970 | 209,952 |
| 6 | 177,100 | 64,000,000 | 52,668 | 3,779,136 |
| 7 | 657,800 | 1,280 × 10⁶ | 201,894 | 68,024,448 |
| 8 | 2,220,075 | 25,600 × 10⁶ | 692,208 | 1,224 × 10⁶ |
| 9 | 6,906,900 | 512,000 × 10⁶ | 2,163,150 | 22,040 × 10⁶ |
| 10 | 20,030,010 | 10,240,000 × 10⁶ | 6,249,100 | 396,719 × 10⁶ |

Compositions are used as a convenient means to build and study the mass distribution of all theoretically possible peptides. Specifically, tryptic peptides with no missed cleavages, up to the mass of 3 kDa are considered, but any other class of peptides or mass limit can be considered by following the same methodology. A useful property of compositions is that one composition codes multiple sequences (Equation (1)) with equal masses. Thus, in order to enumerate all possible peptide masses, it is sufficient to enumerate all amino acid compositions instead of all amino acid sequences.

Still, enumeration of all compositions presents a significant computational challenge given almost exponential growth of their number with length L (Table 1). To address this challenge, a parallel program was developed utilizing a recursive procedure for composition enumeration. The pseudocode of the recursive procedure Generate which generates all compositions of length not greater than L for an alphabet of N letters and prints their masses is as follows:

```
procedure Generate(L, start)
      if start < N then
            for i ← 0 to L
                  c[start] ← i
                  Generate(L − i, start + 1)
      else
            for i ← 0 to L
                  c[N] ← i
                  m ← Mass(c)
                  print m
```

The procedure should be called with parameters (L, 1). Array c holds current composition (n₁, n₂, . . . , n_{N}) and is indexed from one. Procedure Mass returns the mass of the input composition.

Procedure Generate starts with composition (0, 0, . . . , 0) and enumerates all compositions with n_{N} ranging from 0 to L. It then assigns n_{N-1} to 1, and enumerates all compositions with n_{N} ranging from 0 to L-1, and so on. The last composition in this enumeration process is (L, 0, . . . , 0). For example, if N=3 and L=2 then the procedure will enumerate all compositions of length between 0 and 2 in the following way: (0, 0, 0), (0, 0, 1), (0, 0, 2), (0, 1, 0), (0, 1, 1), (0, 2, 0), (1, 0, 0), (1, 0, 1), (1, 1, 0), (2, 0, 0).

Procedure Generate can be made significantly faster by using an upper limit on the mass of compositions. If at some point the mass of the defined components of the current composition exceeds the limit, the further construction of this composition can be canceled. For example, enumeration of all tryptic compositions up to the length 30 takes about 37 hours without an upper mass limit, and about 7 hours with the upper mass limit of 3 kDa (one core, Intel Xeon X5650 CPU).

Execution of procedure Generate in non-parallel fashion is feasible for relatively short lengths L. For example, it takes about 9 minutes to generate the mass distribution of all tryptic peptides up to the length 20 and mass 3 kDa, but this time rises to about 8 hours for all tryptic peptides up to the length 40 (one core, Intel Xeon X5650 CPU). Parallel execution of procedure Generate is based on the fact that a single call to this procedure with parameters (L, 1) can be expanded into L+1 independent calls with start=2: (L, 2), (L−1, 2), . . . , (0, 2), while n₁ is set to 0, 1, . . . , L, correspondingly. Each of these calls, which are called jobs, can be expanded into a set of jobs with start=3, and so on. Note that jobs (L, start+1) and (L−1, start) require less computations than job (L, start).

To generate the mass distribution of all tryptic peptides up to the length 51, the primary problem is split into 229,426 jobs. Table 2 shows the maximum values of parameter L for each value of start that were used in this process. First, the primary job (51, 1) was split into 52 jobs (51, 2), (50, 2), . . . , (0, 2). Then, every job with start=2 and L>20 was split into a set of jobs with start=3. Next, every job with start=3 and L>24 was split into a set of jobs with start=4, and so on; jobs with start=6 were not split into smaller jobs (with start≧7). Parameters listed in Table 2 directly affect computational time and their choice is dependent upon the available computational resources (the number and speed of the CPU cores).

TABLE 2
Maximum values of parameter L used in the job generation.

| start | Lₘₐₓ |
|---|---|
| 2 | 20 |
| 3 | 24 |
| 4 | 28 |
| 5 | 34 |

The general scheme of the computations can be described in the following way. A master process creates a list of jobs for a given primary problem described by parameters (L, 1). It then iterates through the list of jobs and available worker processes and assigns jobs to free workers. A worker generates the mass histogram for the given parameters (L, start) and sends it back to the master. The master adds histograms obtained from the workers to the final mass histogram corresponding to the primary problem. The master and workers can exchange data by using the Message Passing Interface (MPI) [18].

For the mass distribution, compositions are enumerated that correspond to all theoretically possible tryptic peptides without missed cleavages (that is, peptides ending with Lys or Arg, without other Lys and Arg), of length between 2 and 51 amino acids (2≦L≦51), and having the monoisotopic mass no greater than 3 kDa. Note that no tryptic peptides longer than 51 amino acids have the mass of 3 kDa or smaller.

The monoisotopic mass of a (protonated) peptide is calculated as the sum of the monoisotopic masses of its constituent amino acid residues, plus monoisotopic masses of H₂O and a proton. Amino acid masses defined up to the 8th digit after the decimal point are used, and then the peptide mass is rounded to three digits after the decimal points (thus, the mass distribution has accuracy of 0.001 Da). Monoisotopic masses of amino acids that are used herein are:

| Molecule | Monoisotopic Mass |
|---|---|
| H₂O + proton | 19.01784113 |
| G | 57.02146372 |
| A | 71.03711378 |
| S | 87.03202840 |
| P | 97.05276384 |
| V | 99.06841390 |
| T | 101.04767846 |
| C | 103.00918451 |
| L | 113.08406396 |
| I | 113.08406396 |
| N | 114.04292744 |
| D | 115.02694302 |
| Q | 128.05857750 |
| K | 128.09496300 |
| E | 129.04259308 |
| M | 131.04048463 |
| H | 137.05891186 |
| F | 147.06841390 |
| R | 156.10111102 |
| Y | 163.06332852 |
| W | 186.07931294 |

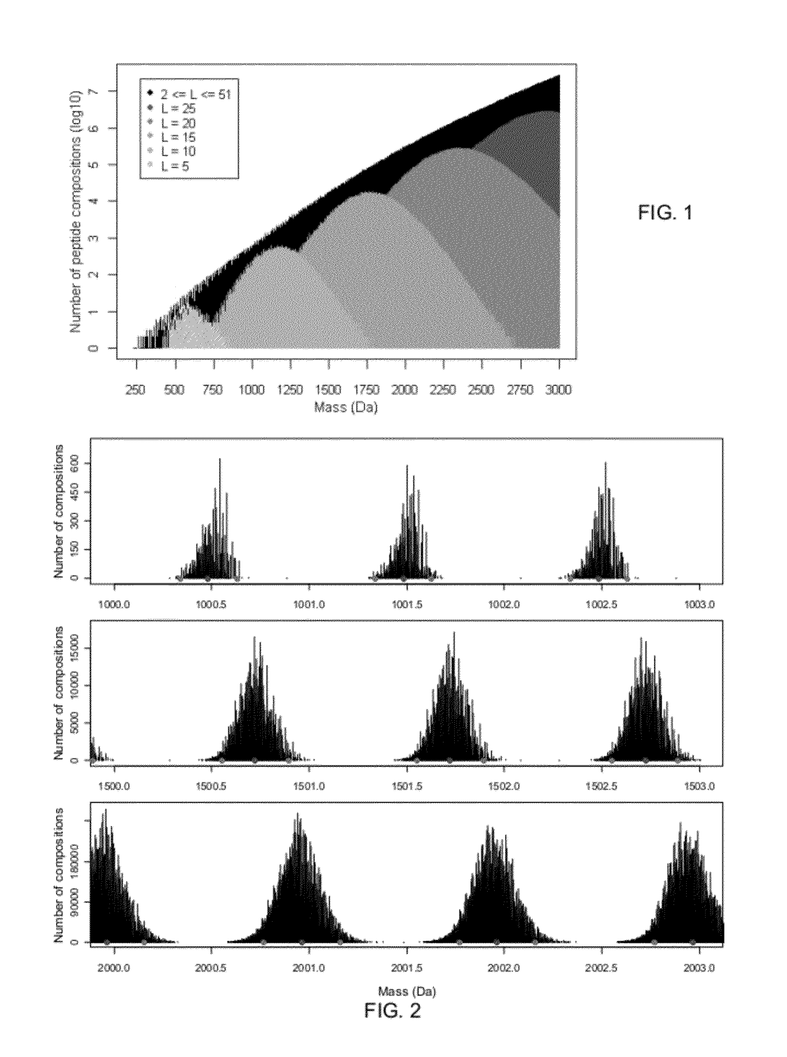

FIG. 1 shows a graph of the logarithmic plot of the number of tryptic peptide compositions versus their mass for 2≦L≦51. Note the almost exponential growth of the number of compositions as the mass increases. Overall, the distribution has about 1.3×10¹² compositions; the lightest peptide, GK, has the mass of 232.140 Da, and the heaviest peptides of 3,000.000 Da are described by 333,571 compositions. The maximum number of compositions, 27,381,800, corresponds to the mass of 2999.399 Da.

Though the mass distribution of compositions for 2≦L≦51 looks solid on the scale of FIG. 1, it actually consists of separate peaks, low populated zones, and gaps as shown in FIG. 2 (red points mark peak centers, green points mark peak widths calculated according to equations (3) and (4)). As the mass increases, these peaks become wider and taller, and their centers move towards the next integer mass value. Mann [12] noticed that for all (not only tryptic) peptides below 2 kDa, the centers of the peaks M_{c} and their widths W_{c} encompassing about 95% of all peptides have the following approximate relationship with the nominal masses Mₙ of peptides:

M_{c}=Mₙ+0.00048Mₙ (3)

W_{c}=0.19+0.0001Mₙ (4)

For the mass range below 2 kDa, it has been determined that 95% of the tryptic peptides lie within the regions described by equations (3) and (4), while for the mass range between 2 and 3 kDa this figure decreases to 90%. For the entire mass range up to 3 kDa this figure is 90% (note that the number of compositions between 2 and 3 kDa is approximately 255 times higher than the number of compositions below 2 kDa).

To characterize the symmetry of the mass peaks, the skewness coefficient [19] was calculated according to the following equation:

$${g}_{1} = \frac{\frac{1}{n} {\sum }_{i = 1}^{n} {( {m}_{i} - \overline{m} )}^{3}}{{( \frac{1}{n} {\sum }_{i = 1}^{n} {( {m}_{i} - \overline{m} )}^{3} )}^{3 / 2}} , \qquad ( 5 )$$

where n is the total number of compositions in a given peak, mᵢ is the mass of the i-th composition, and m̅ is the average mass of all compositions in the peak. Coefficient g₁ is equal to zero for distributions with symmetrical shape, is positive for distributions with longer right tail, and negative for distributions with longer left tail.

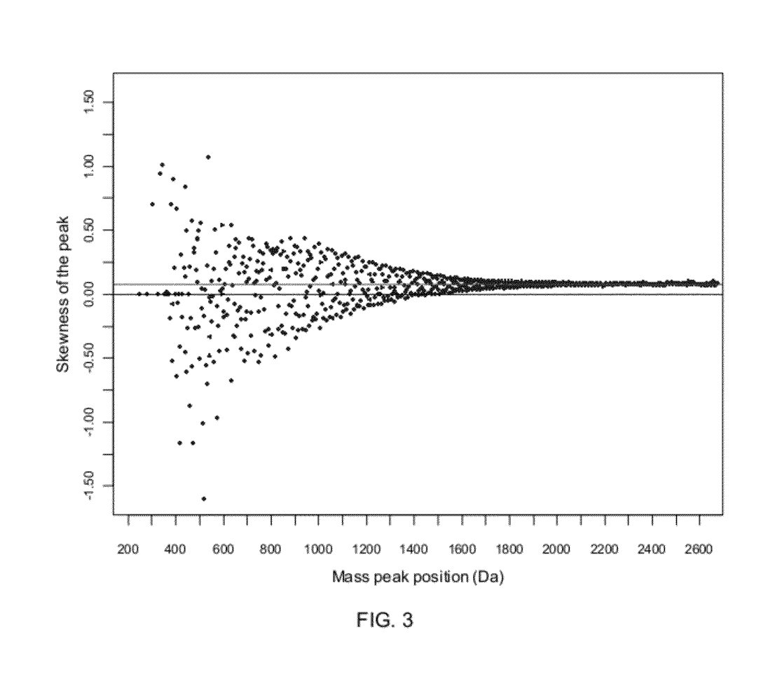

As shown in FIG. 3, for the peaks in the mass distribution of tryptic peptide compositions, coefficient g₁ oscillates around 0.08 with damping amplitude. FIG. 3 is a graph showing the coefficient of skewness calculated for peaks in the mass distribution of all theoretically possible tryptic peptides. Red horizontal line marks value 0.08 approached by the coefficient as the peak position increases. It starts to oscillate between −1.25 to 1.25 for peaks located in the beginning of the mass range, becomes positive for all peaks located above approximately 1500 Da, and oscillates with small constant amplitude after 2000 Da. This means that with increasing mass the shape of the peaks stabilizes and becomes more symmetrical, with slightly longer right tail.

From the practical perspective, an important feature of the distribution of peptide masses is the presence of gaps, which means that for some mass values there exist no amino acid sequences that have these masses. Extended continuous gaps in the distribution of masses of amino acid sequences are called forbidden zones. They can be used to filter out non-peptide masses in a variety of experiments and workflows, including peptide mass fingerprinting' and mass defect filtering [2].

Consider a series of consecutive masses M₁, M₂, M₃, M₄, M₅ and assume that there are 0, 0, 0, K₅ compositions corresponding to these masses, where K₁>0 and K₅>0. Then the gap between M₁ and M₅ can be described by a pair (M₂, 3)—that is, the mass, at which the gap starts, and the width of the gap, measured in the units of the mass scale (Mᵢ−Mᵢ₋₁). If the mass scale unit is 0.001 Da, then the gap's width can be converted into Da by multiplying 3 by 0.001 Da.

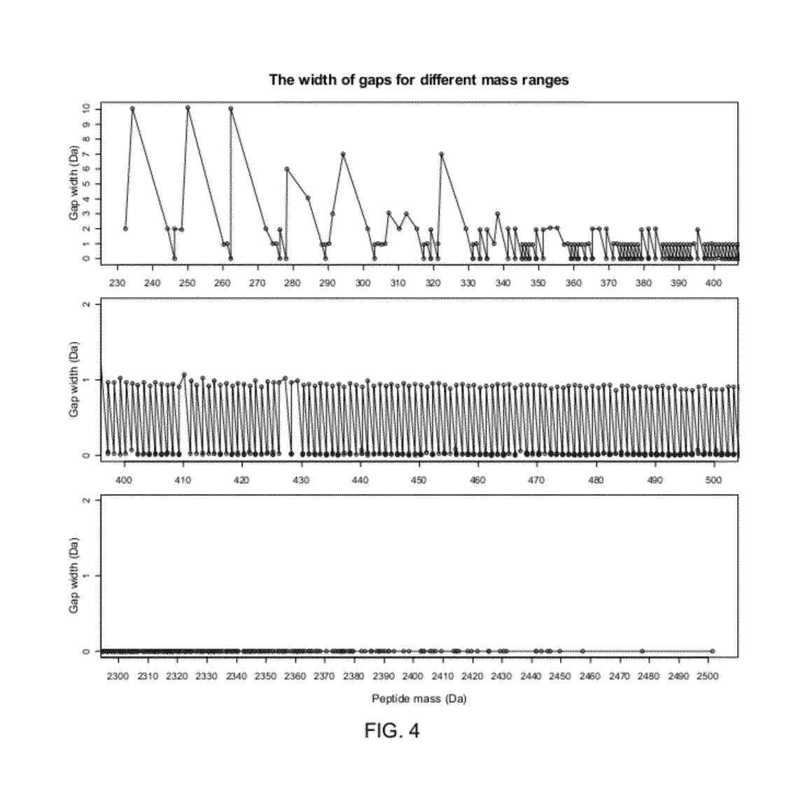

FIG. 4 is a graph that shows gaps' widths for three different mass ranges of the distribution. In the range 230-400 Da there are gaps as wide as 10 Da, due to the boundary effect of the distribution. In the range 400-2,500 Da gap's width varies between 0.001 and 1 Da, and the amplitude of its variation slowly decreases as the mass increases, so that after 1,160 Da the maximum gap is about 0.5 Da; after 1,530 Da it is about 0.25 Da; and after 1752 Da it is about 0.12 Da. The last gap in the distribution is located at mass 2501.517 Da and has width 0.001 Da; after that, the distribution of masses of all theoretically possible tryptic peptides with no missed cleavages (measured at 0.001 Da resolution) has no gaps. Note that the total width of all gaps in the distribution between 232 and 2,502 Da is about 1,203 Da, or 53% of this mass range.

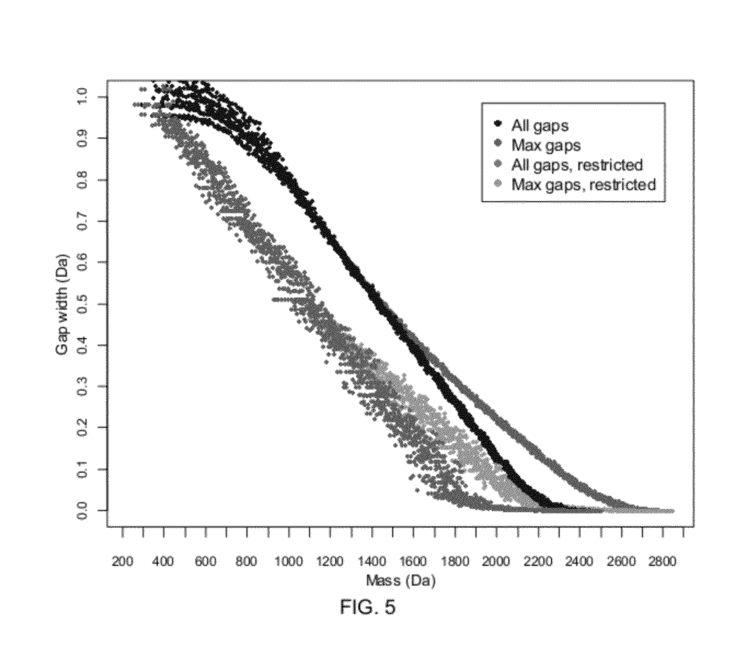

FIG. 5 gives another illustration of how the gaps are closing towards the mass of 2502 Da. Points of dark purple show the total width of all gaps between consecutive peaks, while points of light purple—the width of the maximum continuous gaps between consecutive peaks. Note that the slope of the curves is fairly constant for the most part of the mass range.

As FIG. 2 shows, two consecutive peaks in the mass distribution are separated by gaps and low populated areas, also called quiet zones. It was found that many compositions falling into the quiet zones correspond to sequences with long repetitions of a single amino acid. For example, all compositions in the range 2000.436-2000.445 Da have between 10 and 14 Cys residues, as shown in Table 3. Note long repetitions of amino acid Cys. This particular observation can be explained by the fact that Cys residue has the smallest ratio of the mass defect (the difference between the exact mass and the nominal mass, also referred to as the mass excess [17]) to the monoisotopic mass among all 20 amino acids [2]. Therefore, the right tail of the mass peak located at 1999.96 Da is expected to be populated by the compositions enriched with Cys residues.

TABLE 3
Compositions from quiet zone 2000.436-2000.445 Da.

| | Number | |
|---|---|---|
| | of | |
| Mass (Da) | compositions | Compositions |
| 2000.436 | 1 | P₁C₁₃D₁H₂R |
| 2000.437 | 0 | — |
| 2000.438 | 1 | P₁C₁₁D₄Y₁K |
| 2000.439 | 1 | S₁T₁C₁₃Y₂K |
| 2000.440 | 3 | C₁₃H₁Y₁W₁R, C₁₃N₁W₂R, G₂C₁₃W₂R |
| 2000.441 | 5 | T₁C₁₀D₄M₂K, T₁C₁₁D₂E₂M₁K, |
| | | T₁C₁₂E₄K, S₁C₁₀D₃E₁M₂K, |
| | | S₁C₁₁D₁E₃M₁K |
| 2000.442 | 5 | P₁C₁₁D₂M₃K, P₁C₁₂E₂M₂K, |
| | | S₂C₁₂D₁H₁Y₁R |
| | | S₂C₁₂N₁D₁W₁R, G₂S₂C₁₂D₁W₁R |
| 2000.443 | 2 | V₁C₁₄H₁F₁R, A₁C₁₃M₁H₁F₁R |
| 2000.444 | 0 | — |
| 2000.445 | 2 | S₄C₁₁N₁D₂R, G₂S₄C₁₁D₂R |

To make the mass distribution more realistic, the following restriction was introduced on peptide compositions: every composition (n₁, n₂, . . . , n_{N}) must have nᵢ≦8, i=1, 2, . . . , 20, and n_{L}+n_{L}≦8, where n_{L} is the number of leucines and n₁ is the number of isoleucines. These peptides are rare, which has been confirmed by examination of the IPI human proteome database [20], where only about 1.5% of all tryptic peptides below 3 kDa have single amino acid occurring more than 8 times. Prohibited compositions (about 18% of all compositions) were removed from the original distribution to form the restricted distribution.

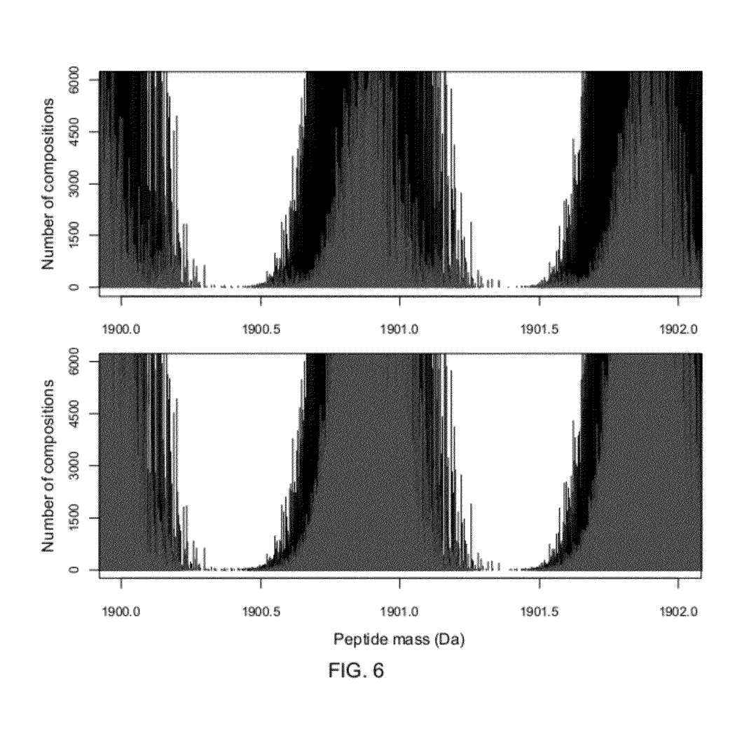

The top graph in FIG. 6 shows a fragment of the non-restricted mass distribution where red peaks correspond to compositions that violate the introduced constraints. It can be see that the proportion of prohibited compositions is larger in the quiet zones than in the peaks. On the bottom graph in FIG. 6, red peaks correspond to uniformly chosen 18% of compositions. It can be seen that the proportion of uniformly chosen compositions is higher in peaks and lower in quiet zones than the proportion of prohibited compositions.

The distribution of gaps' widths for restricted compositions is very similar to that for non-restricted composition, with one anticipated difference: for restricted compositions, the last gap (of 0.001 Da) in the distribution is located at mass 2,842.723 Da, which is about 341 Da further than the last gap in the unrestricted distribution. The total width of all gaps in the restricted distribution between 232 and 2,843 Da is about 1,272 Da, or 49% of this mass range.

FIG. 5 illustrates how the width of the gaps in the restricted distribution changes as the mass increases. Points of dark orange show the total width, and points of light orange—the maximum width of all gaps between consecutive peaks. Note that for the end of the distribution the orange curves have more gentle slopes than purple curves, and they approach zero at larger mass values.

In another experiment, the third mass distribution was produced with restrictions on peptide compositions derived from IPI human protein database (HPD). In silico trypsin digestion of all proteins in HPD were made and calculated length-specific average μᵢⱼ and standard deviation σᵢⱼ of the number of times each AA occurs in tryptic peptides, i=2, 3, . . . , 51 (length index), j=1, 2, . . . , 18 (amino acid index, K and R are excluded). The maximum allowed number of AA j in a composition of length i as μᵢⱼ+2σᵢⱼ was then calculated. For example, the average number of glycines in tryptic peptides of length 4 was 0.22, and the standard deviation of this number was 0.46. Therefore, the maximum allowed number of glycines for compositions of length 4 was set to ceiling(0.22+2*0.46)=2. Prohibited compositions (about 45% of all compositions) were removed from the original distribution to form the restricted distribution.

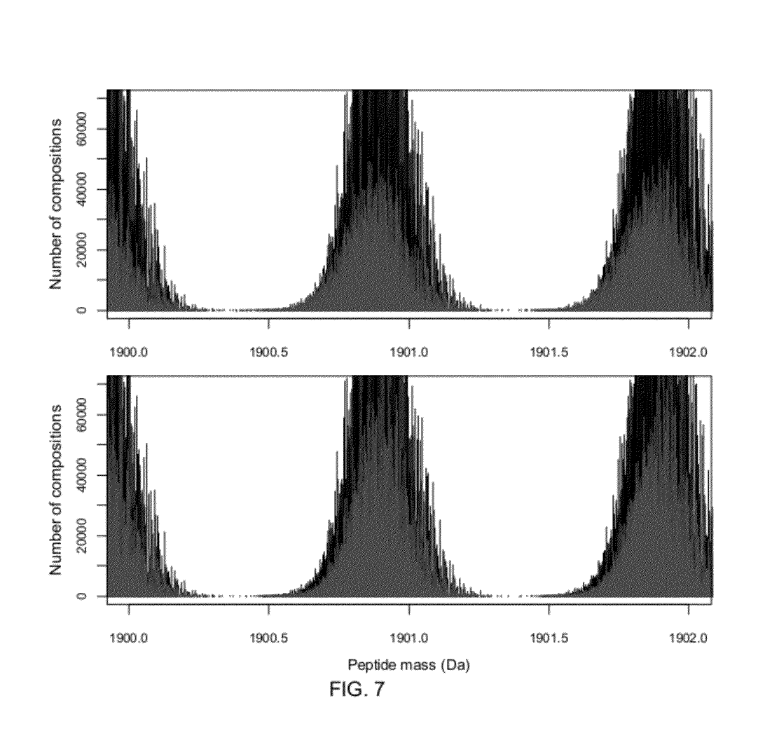

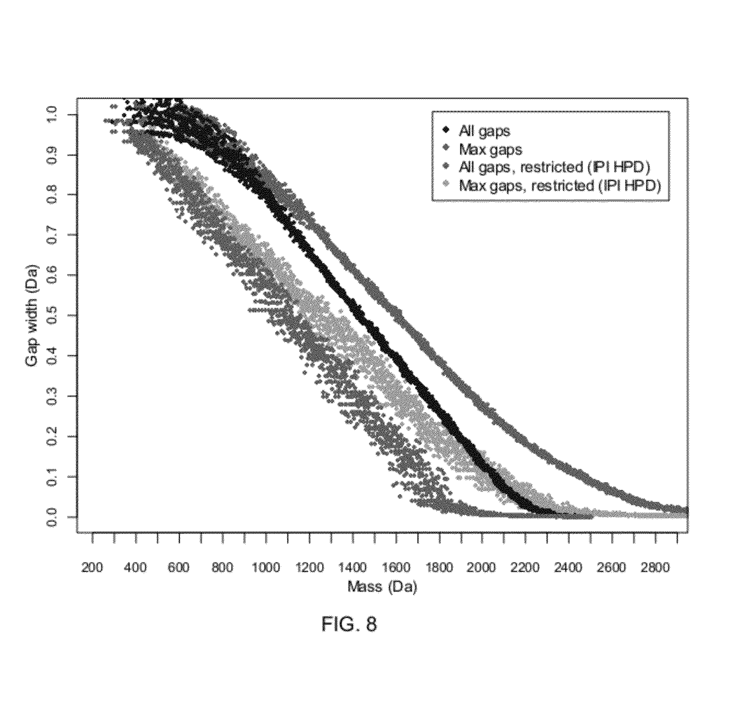

As shown in FIGS. 7 and 8, the effects of restricting number of an amino acid per a composition based on amino acid occurrences in HPD and absolute maximum are similar. The top graph of FIG. 7 shows a fragment of the non-restricted mass distribution with red peaks corresponding to prohibited compositions (according to restrictions derived from the IPI human proteome database) making up 45% of all compositions. The bottom graph of FIG. 7 shows red peaks corresponding to uniformly chosen 45% of all compositions. It can see that the proportion of prohibited compositions is larger in the quiet zones than in the peaks. FIG. 8 is a graph showing a plot of the width of gaps between peaks of the mass distribution. The distribution of gaps for compositions with restrictions derived from the IPI human proteome database has the following features: the last gap (of 0.001 Da) is located at mass 2,999.933 Da; the total width of all gaps in the restricted distribution between 232 and 3,000 Da is about 1,397 Da, or 50% of this mass range. The forbidden zones are extended up to 2200 Da. The all gaps distribution extends somewhat farther with the AA proportions from HPD up to 3 kDa.

To further characterize the diversity of peptide compositions making up peaks and quiet zones of the mass distribution, the average entropy of peptide compositions having the same mass were calculated. For a composition (n₁, n₂, . . . , n_{N}) of length L its entropy is defined as

$$H = - {\sum }_{{n}_{i} > 0} \frac{{n}_{i}}{L} ln ( \frac{{n}_{i}}{L} ) . \qquad ( 6 )$$

As it follows from this equation, for sequences comprised of only one letter, entropy of the corresponding compositions is zero (lowest diversity). On the other hand, for sequences comprised of L different letters, entropy of the corresponding compositions is ln(L) (highest diversity).

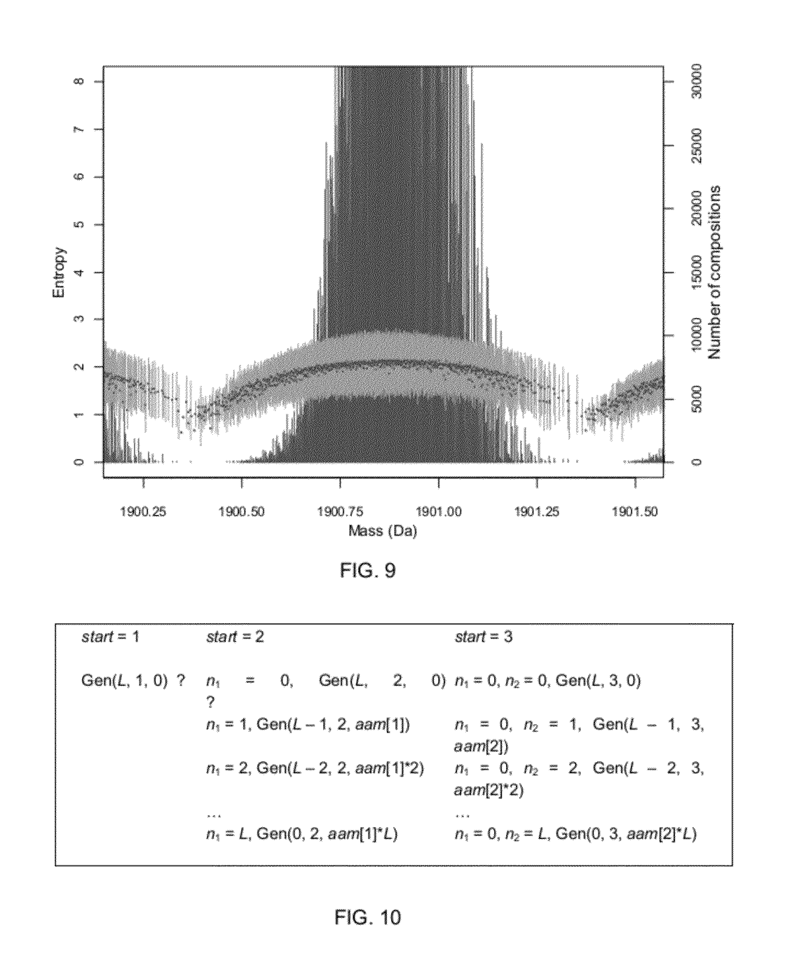

FIG. 9 shows the average entropy of compositions and the corresponding margin of three standard deviations for a short range of mass distribution. The figure demonstrates that the average entropy of compositions is higher in peaks and lower in quiet zones, which means that peaks are made of more diverse compositions than quiet zones. The latter agrees with the observation made above about the distribution of prohibited compositions.

The mass distribution of all theoretically possible tryptic peptides up to the mass of 3 kDa has been described. Specific focus of this study was on characterization of forbidden and quiet zones of the distribution. Mass defect filtering and mass defect labeling require the exact knowledge of the location and width of forbidden zones in the mass distribution of peptides. A detailed description of these zones until they disappear at about 2.5 kDa (for the non-restricted distribution) has been given. An interesting observation is that the total width of all gaps in the distribution constitutes 53% of the mass range between 232 and 2,502 Da.

Analyzing peptide compositions from the quiet zones it was found that many of them correspond to sequences with long repetitions of a single amino acid. This raised the question about realistic (i.e., those that may occur in nature) and nonrealistic peptide sequences, and the probability of specific amino acid patterns occurring. As a first attempt to exclude nonrealistic peptide compositions and generate a more plausible distribution of peptide masses, compositions where each amino acid occurred more than 8 times, or where the cumulative number of leucines and isoleucines was more than 8 were excluded. This rule was justified by examining tryptic peptides obtained from the IPI human protein database. It may be refined by making it dependent on the amino acid, as well as on the length and mass of the peptide.

It was shown that the proportion of prohibited compositions (i.e., containing long repetitions of a single amino acid) is larger in the quiet zones than in the peaks. This was confirmed by the analysis based on entropy of compositions: it was found that entropy (diversity) of the compositions making up peaks of the distribution is higher than entropy (diversity) of compositions making up quiet zones.

The method used to generate this distribution was also described. The method in accordance with the present invention gives substantial reduction in the computation time, allowing all peptide compositions below 3 kDa to be enumerated in 48 minutes on a computer cluster with 12 Intel Xeon X5650 CPUs (72 cores). Fast computation times give an opportunity to routinely generate and use look up tables of all theoretically possible peptide masses in various proteomics experiments. The present invention can be used to enhance the accuracy of protein identification in real mass spectrometry data.

A Parallel Method for enumerating amino acid compositions and masses of all theoretical peptides will now be described. This example describes a parallel method for enumerating all amino acid compositions up to a given length. Recursive procedures are presented, which are at the core of the method, and show that a single task of enumeration of all peptide compositions can be divided into smaller subtasks that can be executed in parallel. The computational complexity of the subtasks is compared with the computational complexity of the whole task. Pseudocodes of processes (a master and workers) that are used to execute the enumerating procedure in parallel are given. Computational times are presented for the method in accordance with the present invention executed on a computer cluster with 12 Intel Xeon X5650 CPUs (72 cores) running Windows HPC Server.

Mass spectrometry (MS) plays a crucial role in modern proteomics as a key method for protein identification and quantification. MS provides accurate mass and abundance measurements of intact and fragmented peptide ions, which are then processed by specialized algorithms and transformed into peptide and protein identities. Thus, efficiency of many MS-based proteomics workflows depends on how well one understands—and can utilize—the properties of peptide masses and peptide mass distribution.

It has been observed that peptide masses have a nonuniform, clustered distribution, which is explained by the fact that peptides are made of twenty amino acids with specific masses. This distribution consists of repeating peaks separated by approximately 1 Da, which become taller and wider as the mass increases. Consecutive peaks are separated by low populated regions (quiet zones) and gaps (forbidden zones)—that is, the mass ranges for which there exist no possible sequences of amino acids. Nonuniformity (peaks, gaps) and discrete nature of the mass distribution of peptides are important for two major problems in MS-based proteomics: peptide identification and de novo sequencing.

The knowledge of the mass distribution of a particular type of peptide (for example, non-modified tryptic peptides) can be used to facilitate peptide identification in a number of ways. Forbidden zones allow us to filter out MS signals corresponding to non-target species (nonpeptide contaminants or modified peptides) early on, before doing any complicated processing of MS data. Dodds and coworkers [1] showed that this results in exponential improvements in statistical significance and discrimination of protein identification based on peptide mass fingerprinting on the Mascot platform. Nonoverlapping or partially overlapping peaks in the mass distributions of different types of peptides allow recognition of these types based solely on precursor masses. For example, Spengler and Hester [21] showed that accurate masses (with accuracy of 0.1 or even 1 ppm) allow phosphorylated and nonmodified peptides to be distinguished. Lehmann and coworkers [22] and Jones and coworkers [23] showed that this is possible for glycopeptides and lipids. In addition, there have been many suggestions for label tags shifting the mass of labeled peptides to quiet or forbidden zones in order to allow easier identification and quantification of these peptides [5].

The major drawback of peptide identification algorithms based on database search is their inability to identify peptides that are not present in the reference database. De novo sequencing algorithms are designed to restore peptide compositions from MS data without the use of peptide databases. These algorithms employ several strategies for MS data analysis [24], one of which is based on the fact that for a given mass there exist only a finite (though sometimes very large) number of amino acid sequences (or amino acid compositions) that can assume that mass, and that these sequences (compositions) can be explicitly enumerated. The use of the masses of fragment ions can further reduce the number of admissible compositions. Several reports have shown the feasibility of this strategy, especially for high accuracy data provided by modern Fourier transform mass spectrometers [6-8].

Proteomics applications mentioned above rely on specific properties of the peptide mass distributions that can only be obtained by enumerating all theoretically possible peptides. Moreover, in many circumstances it is impossible to generate these distributions once and for all, as many parameters can vary from experiment to experiment (peptide modifications, enzymatic specificity, number of missed cleavages, etc.) Thus, it is desirable to be able to generate peptide mass distributions (or some parts of these distributions) “to order” and, therefore, to be able to generate them fast.

Several works focusing on different MS-based proteomics applications employed enumeration of all theoretically possible peptides [8,12,14-16]. Because of the high computational complexity of the task, enumeration of peptides was done for the mass range below 2 kDa, which limited applicability of the obtained results. Also, even for this mass range long computational times and extensive computational capabilities were often required.

The present invention includes a parallel method for enumerating all amino acid compositions up to a given length. First, a pseudocode for recursive procedures is taught. A single task of enumerating all peptide compositions can be divided into smaller subtasks that can be executed in parallel. The computational complexity of these subtasks compares with the computational complexity of the primary task. Finally, pseudocode of processes (a master and workers) is provided that are used to execute the enumerating procedure in parallel. This is the first description of a computational method for a complete and unbiased enumeration of all theoretically possible peptides. The computational times for this method were implemented using Microsoft Visual C++ and the Message Passing Interface (MPI), and executed on a computer cluster with 12 Intel Xeon X5650 CPUs running Windows HPC Server 2008. The mass and length limits are input parameters of the program.

Any peptide composition is represented by a numerical vector (n₁, n₂, . . . , n₂₀), whose i-th component is equal to the number of times the i-th amino acid occurs in the peptide. For example, sequence a₁a₂₀a₁a₁ has composition (3, 0, . . . , 0, 1). In some cases, it is convenient to consider peptides as sequences composed of less or more than 20 letters (tryptic peptides without missed cleavages, post-translationaly modified peptides, etc.). For this reason, a more general notation is adopted: assume an alphabet of N characters and composition vectors (n₁, n₂, . . . , n_{N}). The length of a composition is defined as L=n₁+n₂+ . . . +n_{N}. If mᵢ is the monoisotopic mass associated with the i-th letter, then the monoisotopic mass of a composition is defined as m=n₁m₁+n₂m₂+ . . . +n_{N}m_{N}*. [*The monoisotopic mass of H₂O and a proton may be added to this mass if necessary].

As shown previously in Equations (1) and (2), the number of compositions of length L is equal to the number of ways to choose L elements from a set of N elements if repetitions are allowed. The number of compositions of all lengths not greater than L (including one composition of length 0) is equal to

$${\sum }_{k = 0}^{L} ( \begin{matrix}N + k - 1 \\ k\end{matrix} ) = ( \begin{matrix}N + L \\ N\end{matrix} ) \qquad ( 7 )$$

[27] which follows from the equation

$${\sum }_{k = 0}^{n} ( \begin{matrix}r + k \\ k\end{matrix} ) = ( \begin{matrix}r + n + 1 \\ n\end{matrix} ) . \qquad ( 8 )$$

The latter is based on the recurrence relation

$$( \begin{matrix}r - 1 \\ k\end{matrix} ) + ( \begin{matrix}r - 1 \\ k - 1\end{matrix} ) = ( \begin{matrix}r \\ k\end{matrix} ) , \qquad ( 9 )$$

and can be found in the book by Graham and others [26]. The number of sequences of all lengths not greater than L is equal to

$$\frac{N ( {N}^{L} - 1 )}{N - 1} . \qquad ( 10 )$$

Table 4 shows the number of compositions and sequences for peptides comprised of 20 amino acids. Note how the number of sequences exceeds the number of compositions as the length of peptides grows.

TABLE 4
Number of compositions and sequences comprised of 20 letters, of length
not greater than L, for L ranging from 3 to 10, and their ratios (rounded).

| Length of | Number of Compositions | Number of | |
|---|---|---|---|
| Peptides (L) | (A) | Sequences (B) | Ratio B/A |
| 3 | 1,770 | 8,420 | 5 |
| 4 | 10,625 | 168,420 | 16 |
| 5 | 53,129 | 3,368,420 | 63 |
| 6 | 230,229 | 67,368,420 | 293 |
| 7 | 888,029 | 1,347,368,420 | 1,517 |
| 8 | 3,108,104 | 26,947,368,420 | 8,670 |
| 9 | 10,015,004 | 538,947,368,420 | 53,814 |
| 10 | 30,045,014 | 10,778,947,368,420 | 358,760 |

The pseudocode of a basic recursive procedure for enumerating all compositions of length not greater than L for an alphabet of N letters is as follows:

```
procedure GenBasic(L, start)
      if start < N then
            for i ← 0 to L
                  c[start] ← i
                  GenBasic(L − i, start + 1)
      else
            for i ← 0 to L
                  c[N] ← i
                  m ← Mass(c)
                  print m
```

Array c holds current composition (n₁, n₂, . . . , n_{N}) and is indexed from one. Procedure Mass returns the mass of the input composition c. For a given length L, procedure GenBasic should be called with parameters (L, start=1). Note that the depth of recursion for this procedure is equal to N−1. The number of compositions enumerated by this procedure is given by equation (7).

Procedure GenBasic begins enumeration with composition (0, 0, . . . , 0) and first generate all compositions with n_{N} ranging from 0 to L. It then sets n_{N-1} to 1, and generates all compositions with n_{N} ranging from 0 to L-1, and so on. The last composition in this generation process is (L, 0, . . . , 0). Essentially, the compositions are generated like N-digit numbers, in ascending order, with requirement that the sum of the “digits” must not be greater than L. For instance, for N=3 and L=2 the procedure generates all compositions up to length 2 in the following order: (0, 0, 0), (0, 0, 1), (0, 0, 2), (0, 1, 0), (0, 1, 1), (0, 2, 0), (1, 0, 0), (1, 0, 1), (1, 1, 0), (2, 0, 0).

Several changes to procedure GenBasic will make it faster. First, if L is equal to zero on line 3 then there is no need to make assignment on line 4 and call GenBasic on line 5, since it is already known that the rest of the composition will contain zeros only. Second, the mass of a composition can be calculated as soon as its component nᵢ becomes known, and then pass this mass to the next call of the generating procedure. By doing this, the need to recalculate the mass of the part of the composition that has not been changed is avoided.

The pseudocode of procedure Gen, which is a faster version of procedure GenBasic, is as follows:

```
procedure Gen(L, start, m₀)
comment: global array massHist has been initialized with zero values
if start < N then
      if L = 0 then
            k ← Round(m₀*1000)
            massHist[k] ← massHist[k] + 1
      else
            m ← m₀ − aam[start]
            for i ← 0 to L
                  c[start] ← i
                  m ← m + aam[start]
                  Gen(L − i, start + 1, m)
else
      m ← m₀ − aam[start]
for i ← 0 to L
      c[N] ← i
      m ← m + aam[N]
      k ← Round(m*1000)
      massHist[k] ← massHist[k] + 1
```

Procedure Gen generates a histogram of peptide compositions' masses, instead of printing them, which is more suitable for its further use. The histogram, stored in global array massHist, contains the number of compositions falling into the mass bins of width 0.001 Da. Procedure Round returns the rounded integer value of its argument. Note that since procedure Gen calculates the mass of compositions “on the fly”, compositions do not need to be stored in array c, so lines 10 and 16 may be removed. Assume that array aam of size N stores masses m₁, m₂, . . . , m_{N}. Procedure Gen should be called with parameters (L, start=1, m₀=0).

Enumerating peptide compositions in parallel. The task of enumerating all compositions (n₁, n₂, . . . , n_{N}) can be split into smaller independent subtasks or jobs that can be executed in parallel. Indeed, a single call to procedure Gen with parameters (L, 1, 0) is equivalent to L+1 calls with parameters (L, 2, 0), (L−1, 2, aam[1]), . . . , (0, 2, aam[1]*L), while n₁ is set to 0, 1, . . . , L, correspondingly as shown in FIG. 10. Any job with start=2 can be further expanded into L+1 jobs with start=3, as shown for Gen(L, 2, 0). As before, assume that array aam stores masses m₁, m₂, . . . , m_{N} of the used amino acids. Certainly, the mass histograms produced by each call of procedure Gen will have to be combined, which can be done knowing parameters of each job, described by a triplet (L, start, m₀).

To illustrate this idea, consider again the example with N=3 and L=2. The primary task is to enumerate the following compositions: (0, 0, 0), (0, 0, 1), (0, 0, 2), (0, 1, 0), (0, 1, 1), (0, 2, 0), (1, 0, 0), (1, 0, 1), (1, 1, 0), (2, 0, 0). This can be accomplished by independent enumeration of three subsets of compositions: (i) (0, 0, 0), (0, 0, 1), (0, 0, 2), (0, 1, 0), (0, 1, 1), (0, 2, 0); (ii) (1, 0, 0), (1, 0, 1), (1, 1, 0); and (iii) (2, 0, 0). Compositions (i) can be enumerated by setting n₁=0 and calling Gen with parameters (L=2, start=2, m₀=0); compositions (ii) can be enumerated by setting n₁=1 and calling Gen with parameters (L=1, start=2, m₀=m₁); and single composition (iii) is enumerated by setting n₁=2 and calling Gen with parameters (L=0, start=2, m₀=2m₁).

How can a list or table of jobs be created given the initial job described by parameters (L, 1, 0)? First, job (L, 1, 0) is replaced by L+1 jobs (L, 2, 0), (L−1, 2, aam[1]), . . . , (0, 2, aam[1]*L) (FIG. 10). If, for a given L, job (L, 2, 0) is executed in acceptable time, there is no need to do anything else, and the table of jobs has been initialized. Otherwise, job (L, 2, 0) can be split into L+1 jobs with start=3, and similarly split other jobs with start=2. Thus, for all jobs with start=2 there is certain L_{max,2} such that if the first parameter of the job is larger than L_{max,2} then this job should be split into jobs with start=3. When this is done, move to the jobs with start=3 and process them in a similar manner: all jobs that have first parameter larger than L_{max,3} should be split into jobs with start=4. This is continued until each job in the job table can be executed in acceptable time.

When the table of jobs has been initialized, the jobs from the table can be assigned to computation processes. In this context, it is convenient to think about a master process, which does these assignments, and worker processes, which execute the assigned jobs and return results back to the master. The master then combines partial mass histograms computed by workers into a single final mass histogram. The psuedocode for the master process, procedure CreateMassHistMaster(L) is as follows:

```
procedure CreateMassHistMaster(L)
    create numOfWorkers work processes
    jobs ← CreateJobs(L)
    numOfJobs ← number of created jobs
    numOfBusyWorkers ← 0
    while numOfJobs > 0 or numOfBusyWorkers > 0
        comment: find free process and assign next job
        p ← 1
        while p < numOfProcs and numOfJobs > 0
            if process p is free then
                assign next unassigned job from jobs to process p
                numOfJobs ← numOfJobs − 1
                numOfBusyWorkers ← numOfBusyWorkers + 1
            p ← p + 1
        wait for massHist from any worker
        update massHistGlobal using massHist
        numOfBusyWorkers ← numOfBusyWorkers − 1
    tell work processes to terminate
return massHistGlobal
```

There may be different strategies utilized in assigning the jobs. For example, larger jobs (with larger L) may be assigned prior to smaller jobs (with smaller L). The psuedocode for the worker process, procedure CreateMassHistWorker(L) is as follows:

```
procedure CreateMassHistWorker(L)
      wait for job from the master
      while job is not to terminate
      massHist ← Gen(job.L,job.start,job.m)
            send massHist to the master
            wait for job from the master
      terminate
```

In the experiments, there was no particular strategy in job assignments (jobs were assigned in the order in which they had been inserted into the job table).

The data exchange between the master (lines 11 and 15) and workers (lines 2, 5 and 6) can be organized by using functions MPI_Send and MPI_Receive from any library implementing MPI [18]. In one implementation, Microsoft Visual C++ and MPI library from Microsoft HPC SDK Pack were used.

It is worthwhile to make several additional comments on procedure Gen described above. Various practical considerations may suggest using an upper limit on the mass of peptide compositions that one wants to enumerate. In this case, a significant improvement in computation speed may be achieved by canceling the enumeration of compositions whose mass exceeds a given limit. If array aam contains mass values in ascending order, one can return from function Gen as soon as the current mass (m₀ in line 5, m in lines 11 and 17) exceeds the threshold. To illustrate a possible gain in speed that may be achieved by using a maximum mass limit, consider enumeration of compositions corresponding to all tryptic peptides up to the length of 30. It takes 1 hour 20 minutes to complete the full enumeration of such compositions, while with the mass limit of 3 kDa (heavier peptides are rarely identified in MS experiments) it takes only 11 minutes, as about 87% of the compositions can be skipped (Tables 2 and 3).

There may be other modifications to this procedure, depending on the intended use of the generated mass distribution. For example, the maximum number of occurrences of each amino acid in a peptide may be made limited by a threshold based on the amino acid and the length and/or mass of the peptide. This would make the generated mass distribution more realistic and may increase the lengths of forbidden zones [25]. Instead of counting the number of peptide compositions, one can count the number of peptide sequences using equation (7). In this case, efficient computation of factorials “on the fly” can be implemented similar to the computation of peptide masses. If enzyme-specific peptides are of interest, the procedure can be modified to allow a given number of missed cleavages. The number of amino acids (N) and their monoisotopic masses may vary depending on specific proteases used in sample preparation, possible post-translational or chemical modifications, and other factors. The resolution of the mass histogram (0.001 Da) may be changed as well, without significantly impairing computational speed.

An important question is how the job (L, start+1, 0) compares with the job (L, start, 0) in terms of computational complexity. Let us denote the number of compositions enumerated by the first procedure by C(L, start+1), and the number of compositions enumerated by the second procedure by C(L, start). Using equation (7) provides:

$$\begin{matrix}C ( L , start ) = ( \begin{matrix}N - start + 1 + L \\ N - start + 1\end{matrix} ) . \\ Hence , & ( 11 ) \\ \frac{C ( L , start )}{C ( L , start + 1 )} = 1 + \frac{L}{N - start + 1} . & ( 12 )\end{matrix}$$

For example, if N=20, L=40, and start=1, then C(40, 1)/C(40, 2)=3, which means that a three-fold decrease in computation time is achieved by replacing one call Gen(40, 1, 0) by 41 calls to Gen with start=2, executed in parallel. Similarly,

$$\frac{C ( L , start )}{C ( L - 1 , start )} = 1 + \frac{N - start + 1}{L} . \qquad ( 13 )$$

Thus, if N=20, L=40, and start=2, then C(40, 2)/C(39, 2)≈1.5, which means that Gen(39, 2, 0) will be about 1.5 times faster than Gen(40, 2, 0).

Initialization of a job table requires the maximum value of parameter start, as well as parameters L_{max,2}, L_{max,3}, etc., to be specified. These can be determined empirically based on the available computational resources and the number of processes that can be executed in parallel. For example, it was found that for enumerating tryptic peptide compositions of masses up to 3 kDa by using 72 processes running on 12 Intel Xeon X5650 CPUs the following parameters would give good performance: start≦7, L_{max,2}=20, L_{max,3}=24, L_{max,4}=28, L_{max,5}=34, L_{max,6}=40. The tuning of these parameters is important to ensure good performance, as they directly affect the computation time (Table 5). Computations were done using 71 work processes executed on a cluster with 12 Intel Xeon X5650 CPUs running Windows HPC Server 2008.

TABLE 5
Computation times for enumerating all tryptic compositions up to the length of 30, for different
sets of jobs and number of work processes, with and without the maximum mass limit.

| | | Job Table | | | | | | |
|---|---|---|---|---|---|---|---|---|
| | Number | Number | | | | | | |
| | of | of | | | | | Computation Time | |
| Task | Workers | Jobs | start | L_{max,2} | L_{max,3} | L_{max,4} | massMax = 3 kDa | no massMax |
| L = 30 | 1 | 1 | 1 | — | — | — | 6 h 03 min | 35 h 11 min |
| | 5 | 30 | 2 | — | — | — | 2 h 12 min | 14 h 52 min |
| | 30 | 30 | 2 | — | — | — | 1 h 39 min | 13 h 32 min |
| | 30 | 255 | ≦3 | 20 | — | — | 28 min | 5 h 02 min |
| | 71 | 255 | ≦3 | 20 | — | — | 27 min | 4 h 57 min |
| | 71 | 679 | ≦5 | 20 | 24 | 28 | 11 min | 1 h 20 min |

It should be noted that a job may have jobs with the same parameters L and start, differing only in M. For example, consider the case illustrated in FIG. 7. Splitting job (L, 2, 0) into L+1 jobs with start=3 will give us, among others, job (L−1, 3, aam[2]). On the other hand, splitting job (L−1, 2, aam[1]) into L jobs with start=3 gives us job (L−1, 3, aam[1]). It is clear that execution of these two jobs can be done in one call to function Gen, which should be modified to be able to accept two input masses m₀¹, m₀² instead of m₀, and to work with two variables m¹, m² instead of m. In a similar manner, execution of more than two jobs may be done in one call to function Gen. This approach will lead to a significant speed-up in computations.

In fact, a job table may have jobs with all three parameters L, start and M being equal. Consider, for example, a primary job with L=40, start=0, and m₀=0. Assume that array aam holds amino acid masses in ascending order. Then the first five masses stored in this array will correspond to glycine (G), alanine (A), serine (S), proline (P) and valine (V), and the first five elements of a composition can be denoted by n_{G}, n_{A}, n_{S}, n_{P}, n_{V}. Assume that the job splitting algorithm (see subsection 2.3) yields the following two jobs:

n_{G}=2, n_{A}=0, n_{S}=0, n_{P}=0, n_{V}=1, start=6, L=37,

n_{G}=0, n_{A}=3, n_{S}=0, n_{P}=0, n_{V}=0, start=6, L=37.

Then these two jobs will have the same m₀=213.111 Da, since tripeptides GGV and AAA are oisomeric. If a job table is generated using parameters start≦7, L_{max,2}=20, L_{max,3}=24, L_{max,4}=28, L_{max,5}=34, L_{max,6}=40, then for L=40 about 2% of all jobs will be duplicates; for L=50—about 29%, and for L=60—about 47%. In the case when only the mass distribution of peptide compositions are desired, there is no need to execute duplicate jobs. If certain job occurs k times, it is enough to execute it once and then multiply the resulting histogram by k before adding it to the final histogram. However, if every peptide composition is desired, then duplicate jobs cannot be removed.

Table 6 shows computation times for enumeration of tryptic compositions for a range of lengths between 25 and 55, with and without the use of a maximum mass limit. The numbers in the second column may seem counterintuitive at first, since, for example, it takes 19 min to generate the distribution for L=25 and 11 min for L=35. The explanation, however, lies in using the maximum mass limit of 3 kDa. The longest job for the task with L=25 was L=24, start=2, m₀=0, and it executed for 19 min. The longest job for the task with L=30 was L=24, start=2, m₀=285, and it executed for 8 min. The difference in 11 min comes from the fact that more compositions were canceled out in the second case because of the mass limit that was used. In addition to Table 6, enumeration of all tryptic peptides having the mass no greater than 3 kDa (the length of these peptides does not exceed 51) took 32 minutes. Parameters of the job table were: start≦7, L_{max,2}=20, L_{max,3}=24, L_{max,4}=28, L_{max,5}=34, L_{max,6}=40. Computations were done using 71 work processes executed on a cluster with 12 Intel Xeon X5650 CPUs running Windows HPC Server 2008. Note that these times are only one example and that computation times will vary depending on the number/type of processors and parameters of the job table used.

TABLE 6
Computation times for enumerating all tryptic compositions with different
maximum lengths, with and without maximum mass limit.

| | Computation Time | |
|---|---|---|
| L | maxMass = 3 kDa | no mass limit |
| 25 | 19 min | 29 min |
| 30 | 11 min | 1 h 20 min |
| 35 | 8 min | 5 h 38 min |
| 40 | 8 min | 38 h 28 min |
| 45 | 14 min | >96 h |
| 50 | 29 min | — |

The present invention includes a detailed description of a parallel method for enumerating all theoretically possible amino acid compositions and discussed different aspects of its implementation. Enumeration of all amino acid compositions is important in several proteomics workflows, including peptide mass fingerprinting, mass defect labeling, mass defect filtering, and de novo peptide sequencing. Given the fact that multi-core computers and computer clusters are becoming increasingly available, it is possible to address this computationally expensive task using a parallelization approach.

By reducing computational times from hours to minutes, the applicability of the enumeration of all amino acid compositions in various proteomics studies is significantly improved and extended. The method described herein were used to characterize forbidden and quiet zones in the mass distribution of tryptic peptides [25]. The methods disclosed here can be applied to enhance the accuracy of protein identification in real mass spectrometry data.

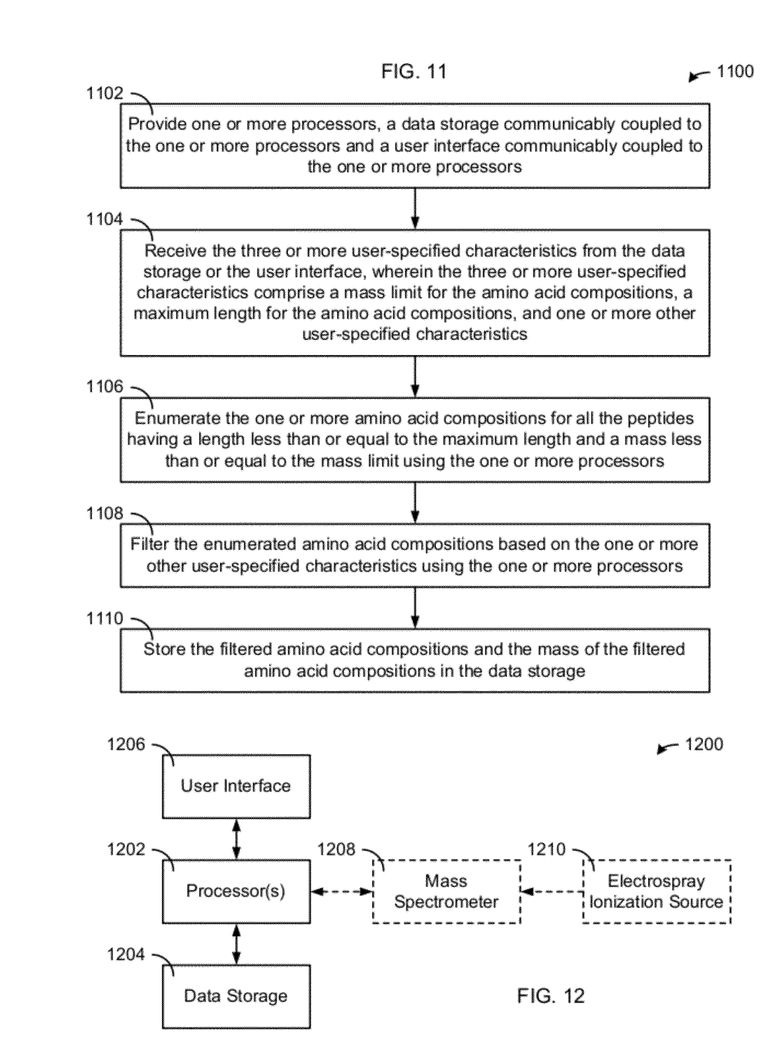

FIG. 11 shows a flow chart of a computerized method 1100 of enumerating one or more amino acid compositions in accordance with one embodiment of the present invention. One or more processors, a data storage communicably coupled to the one or more processors and a user interface communicably coupled to the one or more processors are provided in block 1102. The three or more user-specified characteristics are received from the data storage or the user interface in block 1104. The three or more user-specified characteristics include a mass limit for the amino acid compositions, a maximum length for the amino acid compositions, and one or more other user-specified characteristics. The one or more amino acid compositions are enumerated for all the peptides having a length less than or equal to the maximum length and a mass less than or equal to the mass limit using the one or more processors in block 1106. The enumerated amino acid compositions are filtered based on the one or more other user-specified characteristics using the one or more processors in block 1108. The filtered amino acid compositions and the mass of the filtered amino acid compositions are stored in the data storage in blok 1110. The foregoing method can be implemented as a non-transitory computer-readable medium wherein the steps are executed as one or more code segments by one or more processors.

The one or more other user-specified characteristics may include one or more sample characteristics, one or more experimental parameters, one or more possible post-translational modifications, one or more chemical modifications, one or more types of unrealistic amino acid compositions, zero or more missed cleavages, or one or more mass filters. The one or more possible post-translational modifications may include phosphorylation, methylation, amidation, thiolation, glycosylation, lipidation, non-standard amino acids, ornithine, hydroxyproline or a combination thereof. The one or more chemical modifications may include carbamidomethylation, carboxymethylation or a combination thereof The one or more mass filters may include one or more mass fingerprints, one or more mass defects, or one or more gaps within in a mass distribution. Note also that the mass limit can be a mass range and the maximum length can be a length range.

Additional steps may include: determining the mass of the enumerated amino acid compositions; providing the filtered amino acid compositions to the user interface; generating a histogram of the masses of the filtered amino acid compositions; or determining one or more amino acid sequences for each filtered amino acid composition. Note that the enumeration of the amino acid compositions can be performed by multiple processors operating in parallel. The amino acid sequences can be chosen from at least one of every possible amino acid, every non-standard amino acid, every amino acid and its modification, the twenty most common amino acid residues. The present invention can be used with any mass value range. A mass value in the range of 200 to 3500 Da. will be suitable for many uses of the present invention. In one embodiment, the composition is determined in less than 60, 45, 30, 25, 20, 15, 20, or 10 minutes. In one embodiment, the mass of the composition is about 200 to about 3500 Da, about 200 to about 3000 Da, about 500 to about 1500 Da, about 1000 to about 2000 Da, or about 1500 to about 3000 Da. Moreover, any desired accuracy can be used, but having the enumerated amino acid composition accurate above 0.001 Da. will be suitable for many uses of the present invention.

Another embodiment of the present invention can be used with a mass spectrometer, such that the method also includes the steps of: receiving a mass spectrum for an unknown peptide; determining one or more possible amino acid compositions for the unknown peptide by comparing the mass spectrum to the filtered amino acid compositions using the one or more processors; and providing the possible amino acid compositions to the user interface or storing the possible amino acid compositions in the data storage. In addition, the possible amino acid compositions can be reduced to a limited number of possible amino acid compositions which are consistent with a probability distribution of the mass spectrum. Other steps may include: obtaining the unknown peptide; determining the mass spectrum for the unknown peptide with a mass spectrometer; determining one or more possible amino acid sequences based on the possible amino acid compositions; or determining a probability that the filtered amino acid compositions account for the mass spectrum for the unknown peptide. Note that a portion of the filtered amino acid compositions can have a molecular weight within a predetermined range of the approximate molecular weight of a sample containing the unknown peptide. The predetermined range can be is +/−0.5 Daltons, +/−0.1 Daltons, +/−0.05 Daltons, +/−0.01 Daltons, +/−0.005 Daltons, or +/−0.001 Daltons.

FIG. 12 shows a block diagram of an apparatus 1200 for enumerating one or more amino acid compositions in accordance with another embodiment of the present invention. The apparatus includes one or more processors 1202, a data storage 1204 communicably coupled to the one or more processors 1202, and a user interface 1206 communicably coupled to the one or more processors 1202. The one or more processors (a) receive the three or more user-specified characteristics from the data storage 1204 or the user interface 1206, (b) enumerate the one or more amino acid compositions for all the peptides having a length less than or equal to the maximum length and a mass less than or equal to the mass limit using the one or more processors, (c) filter the enumerated amino acid compositions based on the one or more other user-specified characteristics, and (d) store the filtered amino acid compositions and the mass of the filtered amino acid compositions in the data storage 1204. The three or more user-specified characteristics may include a mass limit for the amino acid compositions, a maximum length for the amino acid compositions, and one or more other user-specified characteristics. Other variations and modifications of the apparatus can be made such as those described above in reference to FIG. 11.

In some embodiments, a mass spectrometer 1208 is communicably connected to the one or more processors 102 for determining the mass spectrum for the unknown peptide with a mass spectrometer. The mass spectrometer can be a tandem mass spectrometer, or a time of flight mass analyzer. An electrospray ionization source 1210 into which the sample containing the unknown peptide is introduced can also be used.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof' is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Dodds, E. D.; An, H. J.; Hagerman, P. J.; Lebrilla, C. B.; Enhanced peptide mass fingerprinting through high mass accuracy: Exclusion of non-peptide signals based on residual mass J. Proteome Res. 2006, 5, 1195-203.

2. Toumi, M. L.; Desaire, H.; Improving mass defect filters for human proteins J. Proteome Res. 2010, 9, 5492-95.

3. Yao, X.; Diego, P.; Ramos, A. A.; Shi, Y.; Averagine-scaling analysis and fragment ion mass defect labeling in peptide mass spectrometry Anal. Chem. 2008, 80, 7383-91.

4. Hernandez, H.; Niehauser, S.; Boltz, S. A.; Gawandi, V.; Phillips, R. S.; Amster, I. J.; Mass defect labeling of cysteine for improving peptide assignment in shotgun proteomic analyses Anal. Chem. 2006, 78, 3417-23.

5. Hall, M. P.; Ashrafi, S.; Obegi, I.; Petesch, R.; Peterson, J. N.; Schneider, L. V.; I. J.; Mass defect tags for biomolecular mass spectrometry Journal of Mass Spectrometry 2003, 38, 809-16.

6. Spengler, B.; Accurate mass as a bioinformatic parameter in data-to-knowledge conversion: Fourier transform ion cyclotron resonance mass spectrometry for peptide de novo sequencing Eur. J. Mass Spectrom. (Chichester, Eng) 2007, 13, 83-87.

7. Spengler, B.; De novo sequencing, peptide composition analysis, and composition-based sequencing: A new strategy employing accurate mass determination by Fourier transform ion cyclotron resonance mass spectrometry Journal of the American Society for Mass Spectrometry 2004, 15, 703-14.

8. Olson, M. T.; Epstein, J. A.; Yergey, A. L.; De novo peptide sequencing using exhaustive enumeration of peptide composition J. Am. Soc. Mass Spectrom. 2006, 17, 1041-49.

9. He, F.; Emmett, M. R.; Hakansson, K.; Hendrickson, C. L.; Marshall, A. G.; Theoretical and experimental prospects for protein identification based solely on accurate mass measurement J. Proteome Res. 2004, 3, 61-67.

10. Mann, M.; Michalski, A.; Cox, J.; More than 100,000 detectable peptide species elute in single shotgun proteomics runs but the majority is inaccessible to data dependent LC MS/MS J. Proteome Res. 2011.

11. Ong, S. E.; Blagoev, B.; Kratchmarova, I.; Kristensen, D. B.; Steen, H.; Pandey, A.; Mann, M.; Stable isotope labeling by amino acids in cell culture, SILAC, as a simple and accurate approach to expression proteomics Mol. Cell Proteomics. 2002, 1, 376-86.

12. Mann, M.; Useful Tables of Possible and Probable Peptide Masses. Annual Conference on Mass Spectrometry and Allied Topics. 5-1-1995. Atlanta, Ga., American Society of Mass Spectrometry. 5-11-1995.

13. Gay, S.; Binz, P. A.; Hochstrasser, D. F.; Appel, R. D.; Modeling peptide mass fingerprinting data using the atomic composition of peptides Electrophoresis 1999, 20, 3527-34.

14. Zubarev, R. A.; Hakansson, P.; Sundqvist, B.; Accuracy requirements for peptide characterization by monoisotopic molecular mass measurements Analytical Chemistry 1996, 68, 4060-63.

15. Demirev, P. A.; Zubarev, R. A.; Probing combinatorial library diversity by mass spectrometry Analytical Chemistry 1997, 69, 2893-900.

16. Fenyo, D.; Qin, J.; Chait, B. T.; Protein identification using mass spectrometric information Electrophoresis 1998, 19, 998-1005.

17. Frahm, J. L.; Howard, B. E.; Heber, S.; Muddiman, D. C.; Accessible proteomics space and its implications for peak capacity for zero-, one- and two-dimensional separations coupled with FT-ICR and TOF mass spectrometry J. Mass Spectrom. 2006, 41, 281-88.

18. Pacheco, P.; Parallel Programming with MPI, 1st ed.; Morgan Kaufman: San Francisco, 1996.

19. Press, W. H.; Numerical recipes in C++ the art of scientific computing, 2nd ed.; Cambridge University Press: Cambridge, UK, 2002.

20. Kersey, P. J.; Duarte, J.; Williams, A.; Karavidopoulou, Y.; Birney, E.; Apweiler, R.; The International Protein Index: an integrated database for proteomics experiments Proteomics. 2004, 4, 1985-88.

21. Spengler, B.; Hester, A.; Mass-Based Classification (MBC) of Peptides: Highly Accurate Precursor Ion Mass Values Can Be Used to Directly Recognize Peptide Phosphorylation. Journal of the American Society for Mass Spectrometry 2008, 19: 1808-1812.

22. Lehmann, W. D.; Bohne, A.; von der Lieth, C. W.; The information encrypted in accurate peptide masses—improved protein identification and assistance in glycopeptide identification and characterization. Journal of Mass Spectrometry 2000, 35: 1335-1341.

23. Jones, J. J.; Stump, M. J.; Fleming, R. C.; Lay, J. O.; Wilkins, C. L.; Strategies and data analysis techniques for lipid and phospholipid chemistry elucidation by intact cell MALDI-FTMS. Journal of the American Society for Mass Spectrometry 2004, 15: 1665-1674.

24. Lu, B.; Chen, T.; Algorithms for de novo peptide sequencing using tandem mass spectrometry. Drug Discovery Today: BIOSILICO 2004, 2: 85-90.

25. Nefedov, A. V.; Mitra, I.; Brasier, A. R.; Sadygov, R. G.; Examining troughs in the mass distribution of all theoretically possible tryptic peptides. J Proteome Res 2011, 10: 4150-4157.

26. Graham, R. L.; Knuth, D. E.; Patashnik, O.; Concrete mathematics: a foundation for computer science, 2nd ed. Reading, Mass: Addison-Wesley; 1994.

27. Nefedov, A. V.; Sadygov, R. G.; A Parallel Method for Enumerating Amino Acid Compositions, BMC Bioinformatics, 2011: 12:432.

28. Cravatt, B. F.; Simon, G. M.; Yates, J. R., III; The biological impact of mass-spectrometry-based proteomics, Nature 2007, 450, 991-1000.

29. Domon, B.; Aebersold, R.; Mass spectrometry and protein analysis, Science 2006, 312, 212-17.

What is claimed is:

1. A computerized method of enumerating one or more amino acid compositions of all theoretically possible peptides having three or more user-specified characteristics, the method comprising the steps of:
providing one or more processors, a data storage communicably coupled to the one or more processors and a user interface communicably coupled to the one or more processors;
receiving the three or more user-specified characteristics from the data storage or the user interface, wherein the three or more user-specified characteristics comprise a mass limit for the amino acid compositions, a maximum length for the amino acid compositions, and one or more other user-specified characteristics;
enumerating the one or more amino acid compositions for all the peptides having a length less than or equal to the maximum length and a mass less than or equal to the mass limit using the one or more processors;
filtering the enumerated amino acid compositions based on the one or more other user-specified characteristics using the one or more processors; and
storing the filtered amino acid compositions and the mass of the filtered amino acid compositions in the data storage.

2. The computerized method as recited in claim 1, wherein the one or more other user-specified characteristics comprise one or more sample characteristics, one or more experimental parameters, one or more possible post-translational modifications, one or more chemical modifications, one or more types of unrealistic amino acid compositions, zero or more missed cleavages, or one or more mass filters.

3. The computerized method as recited in claim 2, wherein the one or more possible post-translational modifications comprise phosphorylation, methylation, amidation, thiolation, glycosylation, lipidation, non-standard amino acids, ornithine, hydroxyproline or a combination thereof

4. The computerized method as recited in claim 2, wherein the one or more chemical modifications comprise carbamidomethylation, carboxymethylation or a combination thereof.

5. The computerized method as recited in claim 2, wherein the one or more mass filters comprise one or more mass fingerprints, one or more mass defects, or one or more gaps within in a mass distribution.

6. The computerized method as recited in claim 1, wherein:
the mass limit comprises a mass range; and
the maximum length comprises a length range.

7. The computerized method as recited in claim 1, further comprising the step of determining the mass of the enumerated amino acid compositions.

8. The computerized method as recited in claim 1, wherein the step of enumerating the amino acid compositions is performed by multiple processors operating in parallel.

9. The computerized method as recited in claim 1, further comprising the step of providing the filtered amino acid compositions to the user interface.

10. The computerized method as recited in claim 1, further comprising the step of generating a histogram of the masses of the filtered amino acid compositions.

11. The computerized method as recited in claim 1, further comprising the step of determining one or more amino acid sequences for each filtered amino acid composition.

12. The computerized method as recited in claim 11, wherein the amino acid sequences are chosen from at least one of every possible amino acid, every non-standard amino acid, or every amino acid and its modification.

13. The computerized method as recited in claim 11, wherein the amino acid sequences are based on the twenty most common amino acid residues.

14. The computerized method as recited in claim 1, wherein the peptides have a mass value in the range of 200 to 3500 Da.

15. The computerized method as recited in claim 1, wherein the enumerated amino acid composition is accurate above 0.001 Da.

16. The computerized method as recited in claim 1, further comprising the steps of:
receiving a mass spectrum for an unknown peptide;
determining one or more possible amino acid compositions for the unknown peptide by comparing the mass spectrum to the filtered amino acid compositions using the one or more processors; and
providing the possible amino acid compositions to the user interface or storing the possible amino acid compositions in the data storage.

17. The computerized method as recited in claim 16, further comprising the step of reducing the possible amino acid compositions to a limited number of possible amino acid compositions which are consistent with a probability distribution of the mass spectrum.

18. The computerized method as recited in claim 16, further comprising the step of determining one or more possible amino acid sequences based on the possible amino acid compositions.

19. The computerized method as recited in claim 16, further comprising the steps of:
obtaining the unknown peptide; and
determining the mass spectrum for the unknown peptide with a mass spectrometer.

20. The computerized method as recited in claim 16, wherein a portion of the filtered amino acid compositions have a molecular weight within a predetermined range of the approximate molecular weight of a sample containing the unknown peptide.

21. The computerized method as recited in claim 20, wherein the predetermined range is +/−0.5 Daltons, +/−0.1 Daltons, +/−0.05 Daltons, +/−0.01 Daltons, +/−0.005 Daltons, or +/−0.001 Daltons.

22. The computerized method as recited in claim 16, further comprising the step of determining a probability that the filtered amino acid compositions account for the mass spectrum for the unknown peptide.

23. An apparatus for enumerating one or more amino acid compositions of all theoretically possible peptides having three or more user-specified characteristics comprising:
one or more processors;
a data storage communicably coupled to the one or more processors;
a user interface communicably coupled to the one or more processors; and
wherein the one or more processors (a) receive the three or more user-specified characteristics from the data storage or the user interface, wherein the three or more user-specified characteristics comprise a mass limit for the amino acid compositions, a maximum length for the amino acid compositions, and one or more other user-specified characteristics, (b) enumerate the one or more amino acid compositions for all the peptides having a length less than or equal to the maximum length and a mass less than or equal to the mass limit using the one or more processors, (c) filter the enumerated amino acid compositions based on the one or more other user-specified characteristics using the one or more processors, and (d) store the filtered amino acid compositions and the mass of the filtered amino acid compositions in the data storage.

24. The apparatus as recited in claim 23, wherein the one or more processors further receive a mass spectrum for an unknown peptide, determine one or more possible amino acid compositions for the unknown peptide by comparing the mass spectrum to the filtered amino acid compositions using the one or more processors, and provide the possible amino acid compositions to the user interface or storing the possible amino acid compositions in the data storage.

25. The apparatus as recited in claim 24, further comprising a mass spectrometer communicably connected to the one or more processors for determining the mass spectrum for the unknown peptide with a mass spectrometer.

26. The apparatus as recited in claim 25, wherein the mass spectrometer comprises a tandem mass spectrometer.

27. The apparatus as recited in claim 25, wherein the mass spectrometer comprises a time of flight mass analyzer.

28. The apparatus as recited in claim 25, further comprising an electrospray ionization source into which the sample containing the unknown peptide is introduced.

29. A non-transitory computer-readable medium for enumerating one or more amino acid compositions of all theoretically possible peptides having three or more user-specified characteristics when executed by one or more processors, the non-transitory computer-readable medium:
a code segment for receiving the three or more user-specified characteristics from the data storage or the user interface, wherein the three or more user-specified characteristics comprise a mass limit for the amino acid compositions, a maximum length for the amino acid compositions, and one or more other user-specified characteristics;
a code segment for enumerating the one or more amino acid compositions for all the peptides having a length less than or equal to the maximum length and a mass less than or equal to the mass limit using the one or more processors;
a code segment for filtering the enumerated amino acid compositions based on the one or more other user-specified characteristics using the one or more processors; and
a code segment for storing the filtered amino acid compositions and the mass of the filtered amino acid compositions in a data storage.
